(19) United States
(12) Patent Application Publication
(10) Pub. No.: US 2026/0115289 A1
(43) Pub. Date: Apr. 30, 2026

(54) LOW DOSE LYMPHODEPLETION WITH ADOPTIVE CELL THERAPY

(71) Applicant: OBSIDIAN THERAPEUTICS, INC., Cambridge, MA (US)

(72) Inventors: Parameswaran Hari, Cambridge, MA (US); Madan Jagasia, Cambridge, MA (US)

(73) Assignee: OBSIDIAN THERAPEUTICS, INC., Cambridge, MA (US)

(21) Appl. No.: 19/374,988

(22) Filed: Oct. 30, 2025

Related U.S. Application Data
(60) Provisional application No. 63/714,568, filed on Oct. 31, 2024.

(51) Int. Cl. A61K 40/11; A61K 31/433; A61K 35/17; A61K 38/51; A61K 40/42; A61K 47/24; A61K 47/26; A61P 35/00

(52) U.S. Cl. CPC A61K 40/11; A61K 31/433; A61K 35/17; A61K 38/51; A61K 40/4234; A61K 47/24; A61K 47/26; A61P 35/00

(57) ABSTRACT

Provided herein is a method of adoptive cell therapy. The method includes administering to a subject a low dose lymphodepletion regimen and administering to the subject a population of tumor infiltrating lymphocytes. Also provided herein is a method of treating cancer in a subject by administering to the subject a low dose lymphodepletion regimen and administering to the subject a population of tumor infiltrating lymphocytes.

### CROSS REFERENCE TO RELATED APPLICATION

This application claims priority to U.S. Provisional Application No. 63/714,568 filed Oct. 31, 2024, which is incorporated by reference herein in its entirety.

### REFERENCE TO A “SEQUENCE LISTING”, A TABLE, OR A COMPUTER PROGRAM LISTING APPENDIX SUBMITTED ON A COMPACT DISK

This application contains a Sequence Listing submitted electronically and is hereby incorporated by reference in its entirety. The Sequence listing.xml file is entitled “108407-1527217-057WO1,” is 16,639 bytes in size, and was created on Oct. 27, 2025.

### BACKGROUND

Investigations in humans and murine models of cancer suggest that lymphodepletion depletes negative regulatory cells including regulatory T cells (Treg cells) and peripheral myeloid-derived suppressor cells, which can suppress T cell proliferation, thus, aiding in the persistence of adoptively transferred cells. Lymphodepleting regimens prior to adoptive cell therapy include, for example, pre-treatment of the recipient subject with full body irradiation or lymphodepleting agents before adoptive cell transfer. Processes involving lymphodepleting agents requires high doses of the agents prior to adoptive cell transfer. Such high doses may cause systemic toxicity and are not well tolerated by patients, making many patients ineligible for adoptive cell therapy. Improvements in the field are needed to make adoptive cell therapy treatments safer and more effective.

### SUMMARY

Provided herein is a method of adoptive cell therapy. The method includes administering to a subject a low dose lymphodepletion regimen for 2 to 4 days, wherein the low dose lymphodepletion regimen comprises administering to the subject 500-1000 mg/m²/day cyclophosphamide and 10-30 mg/m²/day fludarabine and, after administering the low dose lymphodepletion regimen, administering to the subject a population of tumor infiltrating lymphocytes (TIL) modified to express a cytokine which promotes immune cell proliferation (e.g., IL15, including mbIL15, IL7, IL21, IL2, or IL12) operably linked to a drug responsive domain (DRD), wherein the DRD is responsive to a ligand. Optionally, the low dose lymphodepletion regimen comprises administering to the subject 750 mg/m²/day cyclophosphamide for 3 days and 30 mg/m²/day fludarabine for 4 days. Optionally, the method further comprises administering to the subject the ligand (e.g., acetazolamide).

Also provided herein is a method of treating cancer in a subject by administering to the subject a low dose lymphodepletion regimen, wherein the low dose lymphodepletion regimen comprises administering to the subject 500-1000 mg/m²/day cyclophosphamide and 10-30 mg/m²/day fludarabine, and administering to the subject a population of tumor infiltrating lymphocytes modified to express a cytokine which promotes immune cell proliferation (e.g., IL15, including mbIL15, IL7, IL21, IL2, or IL12) operably linked to a DRD, wherein the DRD is responsive to a ligand. Optionally, the low dose lymphodepletion regimen is administered to the subject for 2, 3 or 4 days and prior to administration of the tumor infiltrating lymphocytes. Optionally, the low dose lymphodepletion regimen comprises administering to the subject 750 mg/m²/day cyclophosphamide for 4 days and 30 mg/m²/day fludarabine for 3 days. Optionally, the method further comprises administering to the subject the ligand (e.g., acetazolamide).

### DETAILED DESCRIPTION

Current dogma is that lymphodepletion regimes prior to adoptive cell therapy require high doses of lymphodepleting agents, for example, 60 mg/kg/day (equivalent to 2200 mg/m²/day) cyclophosphamide. Such doses, however, are associated with significant toxicity and, thus, limit the subjects suitable for adoptive cell therapy. The present application provides methods of lymphodepletion and methods of treating cancer comprising a low dose lymphodepletion regimen that can be used to enhance adoptive cell therapies while reducing toxicities associated with using high doses of lymphodepleting agents.

### Methods of Adoptive Cell Therapy

Provided herein are methods of adoptive cell therapy comprising administering to a subject a low dose lymphodepletion regimen for 2 to 4 days, wherein the low dose lymphodepletion regimen comprises 500-1000 mg/m²/day cyclophosphamide and 10-30 mg/m²/day fludarabine and administering to the subject, after the low dose lymphodepletion regimen, a population of tumor infiltrating lymphocytes modified to express a cytokine which promotes immune cell proliferation (e.g., IL15, including mbIL15, IL7, IL21, IL2, or IL12) operably linked to a DRD, wherein the DRD is responsive to a ligand.

As used herein, adoptive cell therapy or adoptive cell transfer refer to a cell therapy involving the transfer of cells into a subject, wherein cells may have originated from the subject or from another individual. The cells may be modified, engineered, or altered, e.g., to express a protein of interest, before being transferred to the patient. By way of example, adoptive cell therapy may comprise administering to the subject a population of tumor infiltrating lymphocytes, as described herein. As used herein, subject and patient are used synonymously and are not meant to be limited to human subjects or patients.

The low dose lymphodepletion regimen used in the methods of adoptive cell therapy herein comprises 500-1000 mg/m²/day cyclophosphamide and 10-30 mg/m²/day fludarabine for 2 to 4 days. Optionally, the subject is administered 500-1000 mg/m²/day cyclophosphamide for 2 days and 10-30 mg/m²/day fludarabine for 2 days. Optionally, the subject is administered 500-1000 mg/m²/day cyclophosphamide for 3 days and 10-30 mg/m²/day fludarabine for 2 days. Optionally, the subject is administered 500-1000 mg/m²/day cyclophosphamide for 4 days and 10-30 mg/m²/day fludarabine for 2 days.

Optionally, the subject is administered 500-1000 mg/m²/day cyclophosphamide for 2 days and 10-30 mg/m²/day fludarabine for 3 days. Optionally, the subject is administered 500-1000 mg/m²/day cyclophosphamide for 3 days and 10-30 mg/m²/day fludarabine for 3 days. Optionally, the subject is administered 500-1000 mg/m²/day cyclophosphamide for 4 days and 10-30 mg/m²/day fludarabine for 3 days.

Optionally, the subject is administered 500-1000 mg/m²/day cyclophosphamide for 2 days and 10-30 mg/m²/day fludarabine for 4 days. Optionally, the subject is administered 500-1000 mg/m²/day cyclophosphamide for 3 days and 10-30 mg/m²/day fludarabine for 4 days. Optionally, the subject is administered 500-1000 mg/m²/day cyclophosphamide for 4 days and 10-30 mg/m²/day fludarabine for 4 days.

Optionally, the subject is administered 500-600 mg/m²/day cyclophosphamide and 10-15 mg/m²/day fludarabine for 2 to 4 days. Optionally, the subject is administered 600-700 mg/m²/day cyclophosphamide and 10-15 mg/m²/day fludarabine for 2 to 4 days. Optionally, the subject is administered 700-800 mg/m²/day cyclophosphamide and 10-15 mg/m²/day fludarabine for 2 to 4 days. Optionally, the subject is administered 800-900 mg/m²/day cyclophosphamide and 10-15 mg/m²/day fludarabine for 2 to 4 days. Optionally, the subject is administered 900-1000 mg/m²/day cyclophosphamide and 10-15 mg/m²/day fludarabine for 2 to 4 days.

Optionally, the subject is administered 500-600 mg/m²/day cyclophosphamide and 15-20 mg/m²/day fludarabine for 2 to 4 days. Optionally, the subject is administered 600-700 mg/m²/day cyclophosphamide and 15-20 mg/m²/day fludarabine for 2 to 4 days. Optionally, the subject is administered 700-800 mg/m²/day cyclophosphamide and 15-20 mg/m²/day fludarabine for 2 to 4 days. Optionally, the subject is administered 800-900 mg/m²/day cyclophosphamide and 15-20 mg/m²/day fludarabine for 2 to 4 days. Optionally, the subject is administered 900-1000 mg/m²/day cyclophosphamide and 15-20 mg/m²/day fludarabine for 2 to 4 days.

Optionally, the subject is administered 500-600 mg/m²/day cyclophosphamide and 20-25 mg/m²/day fludarabine for 2 to 4 days. Optionally, the subject is administered 600-700 mg/m²/day cyclophosphamide and 20-25 mg/m²/day fludarabine for 2 to 4 days. Optionally, the subject is administered 700-800 mg/m²/day cyclophosphamide and 20-25 mg/m²/day fludarabine for 2 to 4 days. Optionally, the subject is administered 800-900 mg/m²/day cyclophosphamide and 20-25 mg/m²/day fludarabine for 2 to 4 days. Optionally, the subject is administered 900-1000 mg/m²/day cyclophosphamide and 20-25 mg/m²/day fludarabine for 2 to 4 days.

Optionally, the subject is administered 500-600 mg/m²/day cyclophosphamide and 25-30 mg/m²/day fludarabine for 2 to 4 days. Optionally, the subject is administered 600-700 mg/m²/day cyclophosphamide and 25-30 mg/m²/day fludarabine for 2 to 4 days. Optionally, the subject is administered 700-800 mg/m²/day cyclophosphamide and 25-30 mg/m²/day fludarabine for 2 to 4 days. Optionally, the subject is administered 800-900 mg/m²/day cyclophosphamide and 25-30 mg/m²/day fludarabine for 2 to 4 days. Optionally, the subject is administered 900-1000 mg/m²/day cyclophosphamide and 25-30 mg/m²/day fludarabine for 2 to 4 days.

Optionally, the low dose lymphodepletion regimen comprises administering to the subject 600-800 mg/m²/day cyclophosphamide for 3 days and 25-30 mg/m²/day fludarabine for 4 days. Optionally, the low dose lymphodepletion regimen comprises administering to the subject 750 mg/m²/day cyclophosphamide for 3 days and 30 mg/m²/day fludarabine for 4 days.

Combinations of doses within the 500-1000 mg/m²/day cyclophosphamide and 10-30 mg/m²/day fludarabine may vary, for example, depending on the subject's weight, height, sex, and the type or severity of the disease for which adoptive cell therapy is administered. Effective amounts and schedules for administering the low dose lymphodepletion regimen may be determined empirically by one skilled in the art based on desired effects and the characteristics of the subject.

The cyclophosphamide and fludarabine as part of the low dose lymphodepletion regimen described herein may be administered by any suitable route. Optionally, the cyclophosphamide and fludarabine are administered by infusion (e.g., intravenously) or orally. Optionally, the low dose lymphodepletion regimen is administered by intravenous infusion or a combination of intravenous infusion and oral administration.

Following the administration of the low dose lymphodepletion regimen, the recipient subject is optionally monitored for the outcome of the lymphodepletion regimen. Thus, for example, the number of Treg cells and potential cytokine sinks can be detected and monitored using methods known in the art. If the desired end point is achieved (e.g., a favorable environment for adoptive cell therapy devoid, or relatively devoid, of Tregs and cytokine sinks), the adoptive cell therapy can proceed.

In the adoptive cell therapy methods herein, the low dose lymphodepletion regimen described above and elsewhere herein is administered to the subject prior to administration of tumor infiltrating lymphocytes modified to express a cytokine which promotes immune cell proliferation (e.g., IL15, including mbIL15, IL7, IL21, IL2, or IL12) operably linked to a DRD, wherein the DRD is responsive to a ligand. As used herein, tumor infiltrating lymphocytes (TIL) refers to a cellular immunotherapy product comprising immune cells (e.g., lymphocytes). Tumor infiltrating lymphocytes can be generated, for example, from a patient's tumor by obtaining immune cells therefrom and subsequently expanding the immune cells ex vivo. The tumor infiltrating lymphocytes may be allogeneic (i.e., the donor subject is not the recipient subject) or autologous (i.e., the donor subject is the recipient subject). Thus, the term tumor infiltrating lymphocytes, as used herein, refers to both autologous and allogeneic tumor infiltrating lymphocytes.

The tumor infiltrating lymphocytes used in the adoptive cell therapy methods described herein may be isolated from a tumor of a donor, wherein the donor subject is the recipient subject (i.e., an autologous source) or is not the recipient subject (i.e., an allogeneic source). The tumor infiltrating lymphocytes can be obtained from a tumor sample via surgical resection, tissue biopsy, needle biopsy or other means. The obtained tumor infiltrating lymphocytes can be selected for reactivity with cancer antigens that are present in the tumor of the recipient subject by methods known in the art, such as Human Leukocyte Antigens (HLA) matching or tetramer staining of the T cell receptor. The adoptive cell therapy method can further comprise selecting a donor subject that is an HLA match for the recipient subject to reduce graft versus host responses.

The tumor infiltrating lymphocytes used in the adoptive cell therapy methods of the present disclosure can be administered by any suitable route. Optionally, the tumor infiltrating lymphocytes are administered by infusion (e.g., intravenous infusion), intraperitoneally, or intrathecally. Optionally, the tumor infiltrating lymphocytes are administered by intravenous infusion. Optionally the tumor infiltrating lymphocytes are administered locally, for example, directly into a tumor or blood vessel that supplies a tumor, into an organ of interest (e.g., the liver), or into a body cavity (e.g., the peritoneum or pleura). The tumor infiltrating lymphocytes can be administered in a single dose or in multiple doses.

The tumor infiltrating lymphocytes used in the adoptive cell therapy methods of the present disclosure are administered to the subject after administration of the low dose lymphodepletion regimen. Optionally, the tumor infiltrating lymphocytes are administered to the subject 1, 2, 3, 4, or 5 days after administration of the low dose lymphodepletion regimen. For example, if the tumor infiltrating lymphocytes are administered at day 0, the low dose lymphodepletion regimen may comprise administering to the subject 750 mg/m²/day cyclophosphamide for 3 days prior to day 0, and 30 mg/m²/day fludarabine for 4 days prior to day 0.

Optionally, the method of adoptive cell therapy comprises administering to the subject a population of tumor infiltrating lymphocytes (TIL) after administration of the low dose lymphodepletion regimen and the subject is not administered exogenous interleukin 2 (IL2). Current protocols for TIL therapy require high-dose IL2 administration beginning on the same day or the day after TIL infusion. In addition to promoting exhaustion of the tumor infiltrating lymphocytes, high doses of IL2 can cause severe side effects in patients with cancer and often cannot be tolerated by those patients in need of adoptive cell therapy. The present methods provide a TIL therapy that optionally requires no exogenous cytokine administration, such as interleukins like IL2, before, during or after administration of the tumor infiltrating lymphocytes. Stated differently, with the present method, there is no need for concomitant IL2 therapy with TIL administration. For example, optionally the subject does not require administration of exogenous IL2 preceding TIL infusion, or for 5 days, 7 days, 10 days, 14 days, 21 days, or 28 days after TIL infusion.

The tumor infiltrating lymphocytes used in the method described herein are modified to express a cytokine which promotes immune cell proliferation (e.g., IL15, IL7, IL21, IL2, or IL12) and then expanded ex vivo to provide a larger population of cells for adoptive cell therapy. The tumor infiltrating lymphocytes modified to express a cytokine which promotes immune cell proliferation have a number of advantages when used in the adoptive cell therapy methods described herein. Namely, the modified tumor infiltrating lymphocytes can be expanded in vitro in the absence of exogenous cytokine and the expanded tumor infiltrating lymphocytes can expand further in vivo without administration of an exogenous cytokine, such as IL2. The result is that the tumor infiltrating lymphocytes are less prone to exhaustion and more able to readily expand and infiltrate the target tumor, which, when combined with the low dose lymphodepletion regimen described herein, improves persistence of the tumor infiltrating lymphocytes.

Optionally, the tumor infiltrating lymphocytes used in the methods described herein is modified to express membrane bound IL15 (mbIL15). IL15, as used herein, refers to an IL15 polypeptide (e.g., UniProtKB-P40933 (IL15_HUMAN)). Optionally, the mbIL15 comprises the amino acid sequence provided in SEQ ID NO: 15 or a polypeptide having at least 85, 86, 87, 88, 89 90, 91, 92, 93, 94 95, 96, 97, 98, 99 or 100% identity to SEQ ID NO: 15 that retains one or more mbIL15 functions (e.g., promoting expansion of modified tumor infiltrating lymphocytes in vivo or promoting cytotoxicity of T and NK cells). IL15 is not generally expressed as a membrane bound molecule, thus, to express mbIL15, the IL15 must be modified to be associated with a transmembrane domain. Thus, the tumor infiltrating lymphocytes may comprise an exogenous nucleic acid sequence that encodes IL15, an exogenous nucleic acid sequence that encodes a transmembrane domain, and, optionally, an exogenous nucleic acid sequence that encodes a linker, hinge, and/or leader sequence. Such an exogenous nucleic acids encoding transmembrane domains or IL15 may be the same or different from nucleic acids naturally present in the tumor infiltrating lymphocytes, but the construct encoding the membrane bound IL15 is not naturally occurring.

Exemplary transmembrane domains that may be used in the method herein optionally comprise an MHC1 transmembrane domain, a CD8 transmembrane domain, a B7-1 transmembrane domain, a CD4 transmembrane domain, a CD28 transmembrane domain, a CTLA-4 transmembrane domain, a PD-1 transmembrane domain, a human IgG4 Fc region, or an IL15 receptor subunit (e.g., IL15αR). The cytokine can be directly linked to the transmembrane domain or may be indirectly connected via a linker or hinge.

Numerous linker sequences (linkers) are known in the art. Linkers include, without limitation, GS linkers, GSG linkers, and GGSG linkers. These linkers are repeats of the subunit one or more times. Thus, a GS linker is a GSₙ linker where n is a numerical number being 1, 2, 3, 4, 5, 6, 7, 8, 9, 10, or more. Similarly, a GSG linker is a GSGₙ linker wherein n is a numerical number being 1, 2, 3, 4, 5, 6, 7, 8, 9, 10, or more. A GGSG linker is a GGSGₙ linker where n is a numerical number being 1, 2, 3, 4, 5, 6, 7, 8, 9, 10 or more. Optionally, the linker used herein is a GS₁₅ linker.

A hinge sequence is a short sequence of amino acids that facilitates flexibility between connected components. The hinge sequence can be any suitable sequence derived or obtained from any suitable molecule. The hinge sequence may be derived from all or part of an immunoglobulin (e.g., IgG1, IgG2, IgG3, IgG4) hinge region, i.e., the sequence that falls between the CH1 and CH2 domains of an immunoglobulin (e.g., an IgG4 Fc hinge), or the extracellular regions of type 1 membrane proteins such as CD8a CD4, CD28, and CD7, which may be a wild type sequence or a derivative thereof. Some hinge regions include an immunoglobulin CH3 domain or both a CH3 domain and a CH2 domain. Optionally, the hinge is derived from a transmembrane domain.

The tumor infiltrating lymphocytes expressing a cytokine which promotes immune cell proliferation (e.g., IL15, including mbIL15, IL7, IL21, IL2, or IL12) described herein optionally further comprise an exogenous nucleic acid sequence that encodes an intracellular/cytoplasmic or transmembrane tail. Optionally, the intracellular/cytoplasmic or transmembrane tail is a B7.1, CD8, CD40L, LIGHT, or NKG2C intracellular tail. The tumor infiltrating lymphocytes expressing a cytokine which promotes immune cell proliferation (e.g., IL15, including mbIL15, IL7, IL21, IL2, or IL12) described herein optionally further comprise an exogenous nucleic acid sequence that encodes a signal sequence (leader sequence). Exemplary leader sequences include

(IGKV1-33; SEQ ID NO: 20)
MDMRVPAQLLGLLLLWLSGARC,
(IgEss; SEQ ID NO: 17)
MDWTWILFLVAAATRVHS,
(Native IL15 LS; SEQ ID NO: 18)
MRISKPHLRSISIQCYLCLLLNSHFLTEAGIHVFILG
CFSAGLPKTEA,
(CD34: SEQ ID NO: 19)
MGLVRRGARAGPRMPRGWTALCLLSLLPSGFMA.

The tumor infiltrating lymphocytes expressing a cytokine which promotes immune cell proliferation (e.g., IL15, including mbIL15, IL7, IL21, IL2, or IL12) described herein are further engineered such that the encoded polypeptide is operably linked to one or more DRDs. Thus, the tumor infiltrating lymphocytes expressing a cytokine which promotes immune cell proliferation (e.g., IL15, including mbIL15, IL7, IL21, IL2, or IL12) described herein may further comprise an exogenous nucleic acid sequence that encodes a DRD.

Drug responsive domains (DRDs) are polypeptides that regulate the expression or activity level of a polypeptide of interest also referred to herein as a payload. DRDs interact with a ligand such that, when the DRD is operatively linked to a payload, it confers ligand-dependent reversible regulation of a characteristic of the payload (for example, activity or abundance). As used herein a payload means any polypeptide (e.g., a cytokine which promotes immune cell proliferation (e.g., IL15, including mbIL15, IL7, IL21, IL2, or IL12) whose function is to be altered by operably linking the payload to DRD according to the disclosure. Operably linked means that the state or function of one polypeptide in the fusion protein is affected by the other polypeptide in the fusion protein. For example, with respect to a fusion protein comprising a DRD and a payload, the DRD and the payload are operably linked if stabilization of the DRD with a ligand results in stabilization of the payload, while destabilization of the DRD in the absence of a ligand results in destabilization of the payload. For example, in the presence of a paired ligand, the DRD, linked to the cytokine directly or indirectly, alters a measurable characteristic of the cytokine (e.g., alters the level of activity or abundance of the cytokine as compared to the level of activity or abundance in the absence of the paired ligand).

The level of amount and/or activity of the cytokine which promotes immune cell proliferation (e.g., IL15, including mbIL15, IL7, IL21, IL2, or IL12) expressed by tumor infiltrating lymphocytes increases in the presence of an effective amount of ligand as compared to the amount or activity in the absence of ligand. An effective amount the ligand means the amount of ligand needed to see an increase in the amount or activity of the cytokine which promotes immune cell proliferation (e.g., IL15, including mbIL15, IL7, IL21, IL2, or IL12). Optionally, the effective amount of the ligand is not so great as to produce unacceptable toxicity or off-target effects. Optionally, the measurable characteristic is a therapeutic outcome, for example, a desired amount or activity of the cytokine which promotes immune cell proliferation (e.g., IL15, including mbIL15, IL7, IL21, IL2, or IL12). By way of example, the amount or activity of the cytokine optionally reduces or eliminates the need for IL2 administration

The DRDs, by way of example, can be chosen from carbonic anhydrase II (CA2, SEQ ID NO: 1), FK506 binding protein (FKBP, SEQ ID NO: 11), E. coli dihydrofolate reductase (ecDHFR, SEQ ID NO: 9), human dihydrofolate reductase (hDHFR, SEQ ID NO: 10), human estrogen receptor (ER, SEQ ID NO: 14), phosphodiesterase 5 (PDE5) full-length (SEQ ID NO: 13), PDE5 ligand binding domain (SEQ ID NO: 12), and or a ligand-binding portion thereof. As used herein, a ligand-binding portion thereof refers to a portion of any of the foregoing DRDs that maintains DRD function, or an amino acid sequence having at least 85%, 86%, 87%, 88%, 89%, 90%, 91%, 92%, 93%, 94%, 95%, 96%, 97%, 98%, 99% or 100% identity to SEQ ID NOs: 1, 3, 5, 7, 9, 10, 11, 12, 13, or 14 or the DRD functional portion thereof. U.S. Pat. Nos. 9,487,787 and 10,137,180, U.S. Publication Nos.: 2019/0192691; 2020/0101142; 2020/0172879; 2021/0069248, and U.S. Pat. No. 17,251,635; and Ser. No. 17/288,373, the contents of each of which are hereby incorporated by reference in their entirety, provide examples of DRDs (and their paired ligands) according to this disclosure. Certain of these and other exemplary DRDs suitable for use according to this disclosure are also provided elsewhere in this specification.

The DRD used in the adoptive cell therapy methods described herein may comprise one or more mutations. One or more mutations (including truncations, substitutions, and deletions or any combination thereof) in the amino acid sequence of CA2, FKBP, ecDHFR, hDHFR, ER, and PDE5, for example, can be advantageous to further destabilize the DRD. Optionally, a DRD of the present disclosure may be derived from CA2 and comprise amino acids 2-260 of the parent CA2 sequence (e.g., amino acids 2-260 of SEQ ID NO: 1). This is referred to herein as a CA2 M1 deletion (M1del) mutation (SEQ ID NO:3). Optionally, a DRD of the present disclosure comprises a region of or the whole human carbonic anhydrase 2, and further comprises one or more mutations relative to the full-length sequence selected from M1del, L156H, and S56N. Optionally, the DRD is selected from the group consisting of SEQ ID NO: 3 (CA2 M1del), SEQ ID NO: 5 (CA2 M1del and L156H) and SEQ ID NO: 7 (CA2 M1del and S56N). The adoptive cell methods described herein comprising administering to the subject a population of tumor infiltrating lymphocytes expressing a cytokine which promotes immune cell proliferation (e.g., IL15, including mbIL15, IL7, IL21, IL2, or IL12) operably linked to a DRD can further comprise administering to the subject a ligand that binds to the DRD in an amount effective to increase the cytokine activity or abundance. The ligand may be any described above and elsewhere herein (e.g., acetazolamide, methotrexate, trimethoprim, shield-1, sildenafil, vardenafil, tadalafil, celecoxib, bazedoxifene or raloxifene). Optionally, the DRD is CA2 and the ligand administered is acetazolamide. The adoptive cell therapy methods described herein optionally further comprise administering to the subject treated with a low dose lymphodepletion a population of tumor infiltrating lymphocytes modified to express a cytokine which promotes immune cell proliferation (e.g., IL15, including mbIL15, IL7, IL21, IL2, or IL12) operably linked to a CA2 DRD and administering to the subject acetazolamide.

Examples of stabilizing ligands and their uses for specific DRDs described herein are shown in Table 1 and are described in U.S. Pat. No. 9,487,787, filed Mar. 33, 2012; U.S. Pat. No. 10,137,180, filed Sep. 6, 2013; PCT Application No. PCT/US2018/037005, filed Jun. 12, 2018; PCT Application No. PCT/US2019/036654, filed Jun. 12, 2019; PCT Application No. PCT/US2019/057698, filed Oct. 23, 2019; PCT Application No. PCT/US2020/021596, filed Mar. 6, 2020; and U.S. application Ser. No. 16/558,224 filed Sep. 2, 2019, the disclosures of all of the aforereferenced applications are incorporated herein by reference in their entireties.

TABLE 1
Listing of DRD and exemplary ligands

| | SEQ ID | |
|---|---|---|
| DRD Protein | NO: | Exemplary Ligands |
| Carbonic anhydrase II (CA2) (Uniprot ID: P00918) | 1 | Celecoxib |
| | | Acetazolamide |
| E. coli dihydrofolate reductase (ecDHFR) (Uniprot | 9 | Methotrexate (MTX) |
| ID: P0ABQ4) | | Trimethoprim (TMP) |
| Human dihydrofolate reductase (hDHFR) (Uniprot | 10 | Methotrexate (MTX) |
| ID: P00374) | | Trimethoprim (TMP) |
| Human FKBP (FK506 binding protein) (Uniprot ID: | 11 | Shield-1 |
| P62942) | | |
| Phosphodiesterase 5 (PDE5), ligand binding domain | 12 | Sildenafil; |
| (Uniprot ID: Uniprot ID O76074) | | Vardenafil; |
| | | Tadalafil |
| Phosphodiesterase 5 (PDE5), full-length (Uniprot | 13 | Sildenafil; |
| ID: Uniprot ID O76074) | | Vardenafil; |
| | | Tadalafil |
| Human estrogen receptor (ER) | 14 | Bazedoxifene |
| Uniprot ID: P03372.2) | | Raloxifene |

The ligand can be administered using a dosing regimen that provides a selected amount a cytokine which promotes immune cell proliferation (e.g., IL15, including mbIL15, IL7, IL21, IL2, or IL12) activity to the subject. The ligand can be delivered to achieve continuous or intermittent cytokine activity in the subject. Determining the frequency and duration of dosing to the subject is determined by a person of skill in the art by considering, for example, providing a higher dose or longer duration of administration of the ligand when more activity of the cytokine which promotes immune cell proliferation (e.g., IL15, including mbIL15, IL7, IL21, IL2, or IL12) is desired and reduces or eliminates the ligand administration when less activity is desired. The dose and duration of ligand administration and the resulting activity of the cytokine is also selected to avoid unacceptable side effects or toxicity in the subject. Thus, the subject is administered an effective amount of the ligand to achieve an effective amount of the cytokine. The term effective amount is defined as any amount necessary to produce a desired physiologic response. Effective amounts and schedules for administering the ligand may be determined empirically by one skilled in the art based on the amount of resulting cytokine, the activity of the cytokine, or based on one or more signs of the effect of the cytokine activity. The ranges for administration of the ligand range from zero to a saturating dose and the resulting cytokine activity ranges from a basal level in the absence of ligand to a maximum level in the presence of a saturating amount of ligand.

The tumor infiltrating lymphocytes modified to express a cytokine which promotes immune cell proliferation (e.g., IL15, including mbIL15, IL7, IL21, IL2, or IL12) operably linked to a DRD may be modified by transducing the tumor infiltrating lymphocytes with a vector having at least a first nucleic acid sequence that encodes the cytokine (e.g., IL15, IL7, IL21, IL2, or IL12) and a second nucleic acid sequence that encodes a transmembrane domain such that, upon expression, the cytokine and transmembrane domain are linked directly or indirectly. Optionally the vector further comprises one or more nucleic acid sequences that encode a signal peptide, a linker, a hinge, an intracellular tail, or a DRD, which, upon expression, are linked directly or indirectly to the cytokine. By way of example, the modified tumor infiltrating lymphocytes can comprise a nucleic acid sequence that encodes a transmembrane domain that is C-terminal to the cytokine polypeptide component and an intracellular tail that is C-terminal to the transmembrane domain. The vector can be configured any number of ways to achieve the desired cytokine operably linked to a DRD. Exemplary nucleic acid constructs are shown in Table 2 below and include the nucleic acid sequences encoding IL15-293 and IL15-292, with and without DRDs, respectively. Thus, the vector optionally comprises the nucleic acid sequence of SEQ ID NOs: 49 or 51. The vector optionally includes or encodes additional elements, such as a promoter sequence and other regulatory elements (enhancers, translational control elements (e.g., IRES), and/or elements that control half-life). The vector optionally comprises or can comprises nucleic acid sequences that encode elements that control translation (e.g., IRES, WPRE, and the like).

The vector can be chosen from viral vectors, plasmids, cosmids, and artificial chromosomes. By way of example, the vector can be a viral vector, such as a lentiviral vector or a retroviral vector. By way of example, the viral vector can a baboon endogenous retrovirus envelope (BaEV) pseudotyped lentiviral vector that comprises a nucleic acid that encodes the and a nucleic acid sequence that encodes a transmembrane domain. Upon expression, the cytokine is associated with the transmembrane domain and is membrane bound by the transmembrane domain. Vectors are optionally transferred to cells by non-viral methods by physical methods such as needles, electroporation, sonoporation, hydroporation; by carriers (such as inorganic particles (e.g., calcium phosphate, silica, gold)), and/or by chemical methods. Optionally, synthetic or natural biodegradable agents are used for delivery such agents including cationic lipids, lipid nano emulsions, nanoparticles, peptide-based vectors, or polymer-based vectors.

Non-limiting examples of constructs and construct components for the modified tumor infiltrating lymphocytes are shown in Table 2. The construct designated IL15-292 includes from the N terminus a signal sequence, IL15, (GS) 15 linker, a hinge region, a transmembrane region, and an intracellular tail. The construct designated IL15-293 includes a DRD (specifically, a CA2 DRD (M1del, L156H)) at the C terminus.

TABLE 2
Examples of TIL constructs and construct components

| | Amino Acid Sequence | | AA SEQ | NA SEQ |
|---|---|---|---|---|
| Description | (AA) | Nucleic Acid Sequence (NA) | ID NO | ID NO |
| IgEss leader | MDWTWILFLVAAATR | — | 17 | — |
| sequence | VHS | | | |
| Native IL 15 | MRISKPHLRSISIQCYL | | 18 | — |
| leader | CLLLNSHFLTEAGIHVF | | | |
| sequence | ILGCFSAGLPKTEA | | | |
| CD34 leader | MGLVRRGARAGPRMP | — | 19 | — |
| sequence | RGWTALCLLSLLPSGF | | | |
| | MA | | | |
| IGKV1-33 | MDMRVPAQLLGLLLL | ATGGACATGCGGGTGCCTGC | 20 | 21 |
| leader | WLSGARC | ACAACTTCTGGGCCTGCTGTT | | |
| sequence | | GTTGTGGCTGTCTGGAGCCC | | |
| | | GGTGT | | |
| Interleukin- | NWVNVISDLKKIEDLI | AATTGGGTAAATGTTATCAG | 22 | 23 |
| 15 (IL15) | QSMHIDATLYTESDVH | TGATCTCAAGAAGATAGAGG | | |
| | PSCKVTAMKCFLLELQ | ATCTCATCCAGTCCATGCAT | | |
| | VISLESGDASIHDTVEN | ATTGATGCCACGCTGTACAC | | |
| | LIILANNSLSSNGNVTE | AGAAAGCGATGTGCATCCTA | | |
| | SGCKECEELEEKNIKEF | GCTGTAAGGTGACAGCGATG | | |
| | LQSFVHIVQMFINTS | AAGTGTTTTCTTTTGGAGCTG | | |
| | | CAGGTAATTAGTCTTGAGTC | | |
| | | CGGCGATGCCAGCATTCATG | | |
| | | ATACCGTAGAAAACTTGATT | | |
| | | ATCCTGGCCAACAATTCTCT | | |
| | | GTCCTCAAACGGAAACGTAA | | |
| | | CCGAGAGCGGTTGTAAAGAA | | |
| | | TGTGAAGAACTGGAAGAAAA | | |
| | | GAACATCAAGGAGTTTCTGC | | |
| | | AATCATTCGTTCACATCGTAC | | |
| | | AAATGTTCATAAATACGTCA | | |
| Linker GS₁₅ | GSGSGSGSGSGSGSGS | GGATCTGGTTCTGGTTCCGG | 24 | 25 |
| | GSGSGSGSGSGSGS | AAGTGGATCTGGTTCAGGGT | | |
| | | CCGGTAGTGGATCTGGGTCA | | |
| | | GGAAGTGGAAGCGGTAGTGG | | |
| | | GTCTGGATCT | | |
| Hinge | KQEHFPDN | AAACAAGAGCACTTTCCTGA | 26 | 27 |
| | | TAAC | | |
| Transmembrane | LLPSWAITLISVNGIFVI | CTGTTGCCGAGCTGGGCGAT | 28 | 29 |
| | CCL | TACGCTTATCAGTGTAAACG | | |
| | | GCATCTTTGTAATATGCTGTC | | |
| | | TG | | |
| Intracellular | TYCFAPRCRERRRNER | ACCTACTGCTTCGCACCAAG | 30 | 31 |
| tail | LRRESVRPV | GTGCCGGGAGAGAAGGAGA | | |
| | | AATGAAAGACTGAGAAGGG | | |
| | | AGAGCGTGAGACCTGTG | | |
| Intracellular | TYCFAPRCRERARNER | ACCTACTGCTTCGCACCAAG | 32 | 33 |
| tail | LRRETVRPV | GTGCCGGGAGAGAGCAAGA | | |
| | | AATGAAAGACTGAGAAGGG | | |
| | | AGACCGTGAGACCTGTG | | |
| Linker (GS) | GS | GGATCC | 34 | 35 |
| ΔU3 - 299- | — | TCTAGAGCGCTTCTGCTCCCC | — | 36 |
| bp deletion | | GAGCTCTATAAAAGAGCCCA | | |
| in the 3′ LTR | | CAACCCCTCACTCGGG | | |
| U3 region; | | | | |
| R in the 3′ | | GCGCCAGTCCTCCGATTGAC | | 37 |
| LTR U3 | | TGAGTCGCCCGGGTACCCGT | | |
| region | | GTATCCAATAAACCCTCTTG | | |
| | | CAGTTGCA | | |
| U5 in the 3′ | — | TCCGACTTGTGGTCTCGCTGT | — | 38 |
| LTR U3 | | TCCTTGGGAGGGTCTCCTCTG | | |
| region | | AGTGATTGACTACCCGTCAG | | |
| | | CGGGGGTCTTTCA | | |
| 3′ LTR U3 | — | TCTAGAGCGCTTCTGCTCCCC | — | 39 |
| region | | GAGCTCTATAAAAGAGCCCA | | |
| | | CAACCCCTCACTCGGGGCGC | | |
| | | CAGTCCTCCGATTGACTGAG | | |
| | | TCGCCCGGGTACCCGTGTAT | | |
| | | CCAATAAACCCTCTTGCAGT | | |
| | | TGCATCCGACTTGTGGTCTCG | | |
| | | CTGTTCCTTGGGAGGGTCTCC | | |
| | | TCTGAGTGATTGACTACCCG | | |
| | | TCAGCGGGGGTCTTTCA | | |
| Packaging | — | TTTGGGGGCTCGTCCGAGAT | — | 40 |
| sequence - Ψ | | CGGAGACCCCTGCCCAGGGA | | |
| psi-region of | | CCACCGACCCACCACCGGGA | | |
| MoMuLV, | | GGTAAGCTGGCCAGCAACTT | | |
| contains | | ATCTGTGTCTGTCCGATTGTC | | |
| splice donor | | TAGTGTCTATGACTGATTTTA | | |
| and B2 | | TGCGCCTGCGTCGGTACTAG | | |
| variant of | | TTAGCTAACTAGCTCTGTATC | | |
| primer | | TGGCGGACCCGTGGTGGAAC | | |
| binding site | | TGACGAGTTCGGAACACCCG | | |
| | | GCCGCAACCCTGGGAGACGT | | |
| | | CCCAGGGACTTCGGGGGCCG | | |
| | | TTTTTGTGGCCCGACCTGAGT | | |
| | | CCTAAAATCCCGATCGTTTA | | |
| | | GGACTCTTTGGTGCACCCCC | | |
| | | CTTAGAGGAGGGATATGTGG | | |
| | | TTCTGGTAGGAGACGAGAAC | | |
| | | CTAAAACAGTTCCCGCCTCC | | |
| | | GTCTGAATTTTTGCTTTCGGT | | |
| | | TTGGGACCGAAGCCGCGCCG | | |
| | | CGCGTCTTGTCTGCTGCAGC | | |
| | | ATCGTTCTGTGTTGTCTCTGT | | |
| | | CTGACTGTGTTTCTGTATTTG | | |
| | | TCTGAAAATGAG | | |
| Ψ + psi+- | | ACTCCCTTAATAAGTTTGACC | — | 41 |
| region of | | TTAGGTCACTGGAAAGTAGT | | |
| MoMuLV | | CGAGCGGATCGCTCACAACC | | |
| (wt), start | | AGTCGGTAGTAGTCAAGAAG | | |
| codon of gag | | AGACGTTGGGTTACCTTCTG | | |
| deleted | | CTCTGCAGAATGGCCAACCT | | |
| | | TTAACGTCGGATGGCCGCGA | | |
| | | GACGGCACCTTTAACCGAGA | | |
| | | CCTCATCACCCAGGTTAAGA | | |
| | | TCAAGGTCTTTTCACCTGGCC | | |
| | | CGCATGGACACCCAGACCAG | | |
| | | GTCCCCTACATCGTGACCTG | | |
| | | GGAAGCCTTGGCTTTTGACC | | |
| | | CCCCTCCCTGGGTCAAGCCC | | |
| | | TTTGTACACCCTAAGCCTCCG | | |
| | | CCTCCTCTTCCTCCATCCGCC | | |
| | | CCGTCTCTCCCCCTTGAACCT | | |
| | | CCTCGTTCGACCCCGCCTCG | | |
| | | ATCCTCCCTTTATCCAGCCCT | | |
| | | CACTCCTTCTCTAGGCGCGCC | | |
| | | C | | |
| MP71 - | — | GCTAGCTTAAGTAACGCCAT | — | 42 |
| promoter | | TTGCAAGGCATGGAAAAATA | | |
| derived from | | CATAACTGAGAATAGAGAAG | | |
| a fragment of | | TTCAGATCAAGGTCAGGAAC | | |
| the | | AGAGAAACAGGAGAATATG | | |
| myeloprolife | | GGCCAAACAGGATATCTGTG | | |
| rative | | GTAAGCAGTTCCTGCCCCGC | | |
| sarcoma | | TCAGGGCCAAGAACAGTTGG | | |
| virus | | AACAGGAGAATATGGGCCAA | | |
| | | ACAGGATATCTGTGGTAAGC | | |
| | | AGTTCCTGCCCCGCTCAGGG | | |
| | | CCAAGAACAGATGGTCCCCA | | |
| | | GATCGGTCCCGCCCTCAGCA | | |
| | | GTTTCTAGAGAACCATCAGA | | |
| | | TGTTTCCAGGGTGCCCCAAG | | |
| | | GACCTGAAATGACCCTGTGC | | |
| | | CTTATTTGAACTAACCAATC | | |
| | | AGTTCGCTTCTCGCTTCTGTT | | |
| | | CGCGCGCTTCTGCTCCCCGA | | |
| | | GCTCAAAAAAAGAGCCCACA | | |
| | | ACCCCTCACTCGGCGCGCCA | | |
| | | GTCCTCCGATAGACTGCGTC | | |
| | | GCCCGGG | | |
| WPRE- | — | AATCAACCTCTGGATTACAA | | 43 |
| Woodchuck | | AATTTGTGAAAGATTGACTG | | |
| Hepatitis | | ATATTCTTAACTATGTTGCTC | | |
| virus post- | | CTTTTACGCTGTGTGGATATG | | |
| transcription | | CTGCTTTAATGCCTCTGTATC | | |
| al regulatory | | ATGCTATTGCTTCCCGTACGG | | |
| element. | | CTTTCGTTTTCTCCTCCTTGT | | |
| | | ATAAATCCTGGTTGCTGTCTC | | |
| | | TTTATGAGGAGTTGTGGCCC | | |
| | | GTTGTCCGTCAACGTGGCGT | | |
| | | GGTGTGCTCTGTGTTTGCTGA | | |
| | | CGCAACCCCCACTGGCTGGG | | |
| | | GCATTGCCACCACCTGTCAA | | |
| | | CTCCTTTCTGGGACTTTCGCT | | |
| | | TTCCCCCTCCCTATCGCCACG | | |
| | | GCAGAACTCATCGCCGCCTG | | |
| | | CCTTGCCCGCTGCTGGACAG | | |
| | | GGGCTAGGTTGCTGGGCACT | | |
| | | GATAATTCCGTGGTGTTGTC | | |
| | | GGGGAAGCTGACGTC | | |
| ΔU3 - 299- | — | AATGAAAGACCCCACCTGTA | | 44 |
| bp deletion | | GGTTTGGCAAGCTAGCTTCT | | |
| in the 5′ LTR | | CGCTTCTGTTCGCGCGCTTCT | | |
| U3 region; | | GCTCCCAGACATCAACAAAA | | |
| | | GAGCCCACAACCCCTCACTC | | |
| | | GGG | | |
| R in the 5′ | — | GCGCCAGTCCTCCGATTGAC | | 45 |
| LTR U3 | | TGAGTCGCCCGGGTACCCGT | | |
| region | | GTATCCAATAAACCCTCTTG | | |
| | | CAGTTGCA | | |
| U5 in the 5′ | — | TCCGACTTGTGGTCTCGCTGT | | 46 |
| LTR U3 | | TCCTTGGGAGGGTCTCCTCTG | | |
| region | | | | |
| | | AGTGATTGACTACCCGTCAG | | |
| | | CGGGGGTCTTTCA | | |
| 5′ LTR U3 | — | AATGAAAGACCCCACCTGTA | — | 47 |
| region | | GGTTTGGCAAGCTAGCTTCT | | |
| | | CGCTTCTGTTCGCGCGCTTCT | | |
| | | GCTCCCAGACATCAACAAAA | | |
| | | GAGCCCACAACCCCTCACTC | | |
| | | GGGGCGCCAGTCCTCCGATT | | |
| | | GACTGAGTCGCCCGGGTACC | | |
| | | CGTGTATCCAATAAACCCTC | | |
| | | TTGCAGTTGCATCCGACTTGT | | |
| | | GGTCTCGCTGTTCCTTGGGA | | |
| | | GGGTCTCCTCTGAGTGATTG | | |
| | | ACTACCCGTCAGCGGGGGTC | | |
| | | TTTCA | | |
| IL15-292 | MDMRVPAQLLGLLLL | ATGGACATGCGGGTGCCTGC | 48 | 49 |
| | WLSGARCNWVNVISD | ACAACTTCTGGGCCTGCTGTT | | |
| | LKKIEDLIQSMHIDATL | GTTGTGGCTGTCTGGAGCCC | | |
| | YTESDVHPSCKVTAM | GGTGTAATTGGGTAAATGTT | | |
| | KCFLLELQVISLESGDA | ATCAGTGATCTCAAGAAGAT | | |
| | SIHDTVENLIILANNSL | AGAGGATCTCATCCAGTCCA | | |
| | SSNGNVTESGCKECEE | TGCATATTGATGCCACGCTG | | |
| | LEEKNIKEFLQSFVHIV | TACACAGAAAGCGATGTGCA | | |
| | QMFINTSGSGSGSGSG | TCCTAGCTGTAAGGTGACAG | | |
| | SGSGSGSGSGSGSGSG | CGATGAAGTGTTTTCTTTTGG | | |
| | SGSGSKQEHFPDNLLP | AGCTGCAGGTAATTAGTCTT | | |
| | SWAITLISVNGIFVICC | GAGTCCGGCGATGCCAGCAT | | |
| | LTYCFAPRCRERRRNE | TCATGATACCGTAGAAAACT | | |
| | RLRRESVRPVGS | TGATTATCCTGGCCAACAAT | | |
| | | TCTCTGTCCTCAAACGGAAA | | |
| | | CGTAACCGAGAGCGGTTGTA | | |
| | | AAGAATGTGAAGAACTGGAA | | |
| | | GAAAAGAACATCAAGGAGTT | | |
| | | TCTGCAATCATTCGTTCACAT | | |
| | | CGTACAAATGTTCATAAATA | | |
| | | CGTCAGGATCTGGTTCTGGTT | | |
| | | CCGGAAGTGGATCTGGTTCA | | |
| | | GGGTCCGGTAGTGGATCTGG | | |
| | | GTCAGGAAGTGGAAGCGGTA | | |
| | | GTGGGTCTGGATCTAAACAA | | |
| | | GAGCACTTTCCTGATAACCT | | |
| | | GTTGCCGAGCTGGGCGATTA | | |
| | | CGCTTATCAGTGTAAACGGC | | |
| | | ATCTTTGTAATATGCTGTCTG | | |
| | | ACCTACTGCTTCGCACCAAG | | |
| | | GTGCCGGGAGAGAAGGAGA | | |
| | | AATGAAAGACTGAGAAGGG | | |
| | | AGAGCGTGAGACCTGTGGGA | | |
| | | TCC | | |
| IL15-293 | MDMRVPAQLLGLLLL | ATGGACATGCGGGTGCCTGC | 50 | 51 |
| | WLSGARCNWVNVISD | ACAACTTCTGGGCCTGCTGTT | | |
| | LKKIEDLIQSMHIDATL | GTTGTGGCTGTCTGGAGCCC | | |
| | YTESDVHPSCKVTAM | GGTGTAATTGGGTAAATGTT | | |
| | KCFLLELQVISLESGDA | ATCAGTGATCTCAAGAAGAT | | |
| | SIHDTVENLIILANNSL | AGAGGATCTCATCCAGTCCA | | |
| | SSNGNVTESGCKECEE | TGCATATTGATGCCACGCTG | | |
| | LEEKNIKEFLQSFVHIV | TACACAGAAAGCGATGTGCA | | |
| | QMFINTSGSGSGSGSG | TCCTAGCTGTAAGGTGACAG | | |
| | SGSGSGSGSGSGSGSG | CGATGAAGTGTTTTCTTTTGG | | |
| | SGSGSKQEHFPDNLLP | AGCTGCAGGTAATTAGTCTT | | |
| | SWAITLISVNGIFVICC | GAGTCCGGCGATGCCAGCAT | | |
| | LTYCFAPRCRERRRNE | TCATGATACCGTAGAAAACT | | |
| | RLRRESVRPVGSSHHW | TGATTATCCTGGCCAACAAT | | |
| | GYGKHNGPEHWHKDF | TCTCTGTCCTCAAACGGAAA | | |
| | PIAKGERQSPVDIDTHT | CGTAACCGAGAGCGGTTGTA | | |
| | AKYDPSLKPLSVSYDQ | AAGAATGTGAAGAACTGGAA | | |
| | ATSLRILNNGHAFNVE | GAAAAGAACATCAAGGAGTT | | |
| | FDDSQDKAVLKGGPL | TCTGCAATCATTCGTTCACAT | | |
| | DGTYRLIQFHFHWGSL | CGTACAAATGTTCATAAATA | | |
| | DGQGSEHTVDKKDSIK | CGTCAGGATCTGGTTCTGGTT | | |
| | TKGKSADFTNFDPRGL | CCGGAAGTGGATCTGGTTCA | | |
| | LPESLDYWTYPGSLTT | GGGTCCGGTAGTGGATCTGG | | |
| | PPLLECVTWIVLKEPIS | GTCAGGAAGTGGAAGCGGTA | | |
| | VSSEQVLKFRKLNFNG | GTGGGTCTGGATCTAAACAA | | |
| | EGEPEELMVDNWRPA | GAGCACTTTCCTGATAACCT | | |
| | QPLKNRQIKASFK | GTTGCCGAGCTGGGCGATTA | | |
| | | CGCTTATCAGTGTAAACGGC | | |
| | | ATCTTTGTAATATGCTGTCTG | | |
| | | ACCTACTGCTTCGCACCAAG | | |
| | | GTGCCGGGAGAGAAGGAGA | | |
| | | AATGAAAGACTGAGAAGGG | | |
| | | AGAGCGTGAGACCTGTGGGA | | |
| | | TCCTCCCATCACTGGGGGTA | | |
| | | CGGCAAACACAACGGACCTG | | |
| | | AGCACTGGCATAAGGACTTC | | |
| | | CCCATTGCCAAGGGAGAGCG | | |
| | | CCAGTCCCCTGTTGACATCG | | |
| | | ACACTCATACAGCCAAGTAT | | |
| | | GACCCTTCCCTGAAGCCCCT | | |
| | | GTCTGTTTCCTATGATCAAGC | | |
| | | AACTTCCCTGAGAATCCTCA | | |
| | | ACAATGGTCATGCTTTCAAC | | |
| | | GTGGAGTTTGATGACTCTCA | | |
| | | GGACAAAGCAGTGCTCAAGG | | |
| | | GAGGACCCCTGGATGGCACT | | |
| | | TACAGATTGATTCAGTTTCAC | | |
| | | TTTCACTGGGGTTCACTTGAT | | |
| | | GGACAAGGTTCAGAGCATAC | | |
| | | TGTGGATAAAAAGAAATATG | | |
| | | CTGCAGAACTTCACTTGGTTC | | |
| | | ACTGGAACACCAAATATGGG | | |
| | | GATTTTGGGAAAGCTGTGCA | | |
| | | GCAACCTGATGGACTGGCCG | | |
| | | TTCTAGGTATTTTTTTGAAGG | | |
| | | TTGGCAGCGCTAAACCGGGC | | |
| | | CATCAGAAAGTTGTTGATGT | | |
| | | GCTGGATTCCATTAAAACAA | | |
| | | AGGGCAAGAGTGCTGACTTC | | |
| | | ACTAACTTCGATCCTCGTGG | | |
| | | CCTCCTTCCTGAATCCCTGGA | | |
| | | TTACTGGACCTACCCAGGCT | | |
| | | CACTGACCACCCCTCCTCTTC | | |
| | | TGGAATGTGTGACCTGGATT | | |
| | | GTGCTCAAGGAACCCATCAG | | |
| | | CGTCAGCAGCGAGCAGGTGT | | |
| | | TGAAATTCCGTAAACTTAAC | | |
| | | TTCAATGGGGAGGGTGAACC | | |
| | | CGAAGAACTGATGGTGGACA | | |
| | | ACTGGCGCCCAGCTCAGCCA | | |
| | | CTGAAGAACAGGCAAATCAA | | |
| | | AGCTTCCTTCAAA | | |

### Methods of Treating Cancer

Also provided herein is a method of treating cancer in a subject by administering to the subject a low dose lymphodepletion regimen, wherein the low dose lymphodepletion regimen comprises administering to the subject 500-1000 mg/m²/day cyclophosphamide and 10-30 mg/m²/day fludarabine, and administering to the subject a population of tumor infiltrating lymphocytes modified to express a cytokine which promotes immune cell proliferation (e.g., IL15, including mbIL15, IL7, IL21, IL2, or IL12) operably linked to a DRD, wherein the DRD is responsive to a ligand. Optionally, the low dose lymphodepletion regimen is administered for 2 to 4 days (e.g., 2, 3, or 4 days) and prior to administration of the population of tumor infiltrating lymphocytes. Optionally, the tumor infiltrating lymphocytes are administered to the subject 1, 2, 3, 4, or 5 days after administration of the low dose lymphodepletion regimen. For example, if the tumor infiltrating lymphocytes are administered at day 0, the low dose lymphodepletion regimen may comprise administering to the subject 750 mg/m²/day cyclophosphamide for 3 days prior to day 0, and 30 mg/m²/day fludarabine for 4 days prior to day 0.

As used herein, the term cancer refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. Optionally, the methods of treating cancer provided herein are used to treat a solid cancer. For example, the present methods may be used to treat head and neck squamous cell carcinoma, melanoma, lung cancer, or genitourinary cancers.

The recipient subject is optionally monitored for the outcome of the cancer treatment. For example, the number of malignant cells in a sample, the circulating tumor DNA in a sample, or the size of a solid tumor upon imaging can be detected. If the desired end point is achieved (e.g., showing successful treatment of cancer), the ligand can be reduced or discontinued so as to reduce or eliminate the amount or activity of the cytokine. Similarly, if the subject develops a cytokine storm, an allergic reaction, or other adverse effect from the cytokine, the ligand can be reduced or discontinued. Administration of the ligand can optionally be increased or restarted after the reduction or discontinuation. The above-described low dose lymphodepletion regimen also applies to the present methods of treating cancer. For example, the method of treating cancer optionally includes the low dose lymphodepletion regimen described herein.

Additionally, the above-described methods of preparing tumor infiltrating lymphocytes and features of tumor infiltrating lymphocytes apply to the population of tumor infiltrating lymphocytes used in the present methods of treating cancer. The population of tumor infiltrating lymphocytes used in the methods of treating cancer is modified such that at least a portion of the population of tumor infiltrating lymphocytes express a cytokine which promotes immune cell proliferation (e.g., IL15, including mbIL15, IL7, IL21, IL2, or IL12) operably linked to a DRD. The tumor infiltrating lymphocytes may be administered to the subject without IL2 administration and with administration by known methods in the art, including those described above and elsewhere herein.

The present methods of treating cancer may further comprise administering to the subject a ligand that binds to the DRD in an amount effective to increase the activity of the cytokine which promotes immune cell proliferation (e.g., IL15, including mbIL15, IL7, IL21, IL2, or IL12). The ligand may be any described above and elsewhere herein (e.g., acetazolamide, methotrexate, trimethoprim, shield-1, sildenafil, vardenafil, tadalafil, celecoxib, bazedoxifene or raloxifene). Dosages and routes for ligand administration are known in the art and discussed above. Optionally, the DRD is CA2 and the ligand administered is acetazolamide

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference and understanding, and the inclusion of such definitions herein should not necessarily be construed to mean a substantial difference over what is generally understood in the art. Commonly understood definitions of molecular biology terms and/or methods and/or protocols can be found in Rieger et al., Glossary of Genetics: Classical and Molecular, 5th edition, Springer-Verlag: New York, 1991; Lewin, Genes V, Oxford University Press: New York, 1994; Sambrook et al., Molecular Cloning, A Laboratory Manual (3d ed. 2001) and Ausubel et al., Current Protocols in Molecular Biology (1994), Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Publications cited herein and the material for which they are cited are hereby specifically incorporated by reference in their entireties.

### EXAMPLES

The examples below are intended to further illustrate certain aspects of the methods and compositions described herein and are not intended to limit the scope of the claims.

### Example 1. Low Dose Lymphodepletion Regimen

Subjects were administered either a standard dose or a low dose lymphodepletion regimen. The standard dose lymphodepletion regimen involved administering to the subject 60 mg/kg/day (equivalent to 2,200 mg/m²/day) cyclophosphamide for two days, and 25 mg/m²/day fludarabine for five days prior to adoptive cell therapy of administration of tumor infiltrating lymphocytes expressing mbIL15 operably linked to a CA2 DRD followed by ligand (e.g., acetazolamide) administration. The low dose lymphodepletion regimen involved administering to the subject 750 mg/m²/day cyclophosphamide for three days, and 30 mg/m²/day fludarabine for four days prior to adoptive cell therapy and ligand administration.

Subjects receiving the low dose lymphodepletion regimen before tumor infiltrating lymphocytes administration were observed to exhibit proliferation and persistence of the infused tumor infiltrating lymphocytes.

What is claimed is:

1. A method of adoptive cell therapy, comprising:
(a) administering to a subject a low dose lymphodepletion regimen for 2 to 4 days, wherein the low dose lymphodepletion regimen comprises 500-1000 mg/m²/day cyclophosphamide and 10-30 mg/m²/day fludarabine; and
(b) administering to the subject after step (a) a population of tumor infiltrating lymphocytes, wherein the tumor infiltrating lymphocytes are modified to express a membrane-bound IL15, wherein the membrane-bound IL15 is operably linked to a drug responsive domain (DRD), and wherein the DRD is responsive to a ligand.

2. The method of claim 1, wherein the low dose lymphodepletion regimen is administered for 4 days.

3. The method of claim 1, wherein the low dose lymphodepletion regimen comprises 750 mg/m²/day cyclophosphamide for 3 days and 30 mg/m²/day fludarabine for 4 days.

4. The method of claim 1, wherein the low dose lymphodepletion regimen and the population of tumor infiltrating lymphocytes are administered without IL2.

5. The method of claim 1, wherein the DRD is derived from a protein selected from the group consisting of carbonic anhydrase II (CA2, SEQ ID NO: 1); FK506 binding protein (FKBP, SEQ ID NO:11), E. coli dihydrofolate reductase (ecDHFR, SEQ ID NO: 9), human dihydrofolate reductase (hDHFR, SEQ ID NO: 10), human estrogen receptor (ER, SEQ ID NO: 14), phosphodiesterase 5 (PDE5) full-length (SEQ ID NO: 13), PDE5 ligand binding domain (SEQ ID NO: 12), and or a ligand-binding portion thereof.

6. The method of claim 1, further comprising administering to the subject the ligand.

7. The method of claim 1, wherein the DRD is derived from carbonic anhydrase II (CA2, SEQ ID NO: 1) and the ligand is acetazolamide.

8. A method of treating cancer in a subject, comprising
(a) administering to the subject a low dose lymphodepletion regimen, wherein the low dose lymphodepletion regimen comprises 500-1000 mg/m²/day cyclophosphamide and 10-30 mg/m²/day fludarabine; and
(b) administering to the subject a population of tumor infiltrating lymphocytes, wherein the tumor infiltrating lymphocytes are modified to express a membrane-bound IL15, wherein the membrane-bound IL15 is operably linked to a drug responsive domain (DRD), and wherein the DRD is responsive to a ligand.

9. The method of claim 8, wherein the low dose lymphodepletion regimen is administered to the subject for 2 to 4 days and prior to administration of the population of tumor infiltrating lymphocytes.

10. The method of claim 9, wherein the low dose lymphodepletion regimen is administered for 4 days.

11. The method of claim 8, wherein the low dose lymphodepletion regimen comprises 750 mg/m²/day cyclophosphamide for 4 days and 30 mg/m²/day fludarabine for 3 days.

12. The method of claim 8, wherein the low dose lymphodepletion regimen and the population of tumor infiltrating lymphocytes are administered without IL2.

13. The method of claim 8, wherein the DRD is derived from a protein selected from the group consisting of carbonic anhydrase II (CA2, SEQ ID NO: 1); FK506 binding protein (FKBP, SEQ ID NO:11), E. coli dihydrofolate reductase (ecDHFR, SEQ ID NO: 9), human dihydrofolate reductase (hDHFR, SEQ ID NO: 10), human estrogen receptor (ER, SEQ ID NO: 14), phosphodiesterase 5 (PDE5) full-length (SEQ ID NO: 13), PDE5 ligand binding domain (SEQ ID NO: 12), and or a ligand-binding portion thereof.

14. The method of claim 8, further comprising administering to the subject the ligand.

15. The method of claim 8, wherein the DRD is derived from carbonic anhydrase II (CA2, SEQ ID NO: 1) and the ligand is acetazolamide.


SEQUENCE LISTING (12 sequences)

SEQ ID NO: 1 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 2 - DNA, 1492 bp, Homo sapiens
  source 1..1492: mol_type = genomic DNA
acacagtgcaggcgcccaagccgccgccgccagatcggtgccgattcctgccctgccccgaccgccagcgcgaccatgtcccatcactgggggtacggcaaacacaacggacctgagcactggcataaggacttccccatcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccttggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataagatggtcccatagtctgtatccaaataatgaatcttcgggtgtttccctttagctaagcacagatctaccttggtgatttggaccctggttgctttgtgtctagttttctagacccttcatctcttacttgatagacttactaataaaatgtgaagactagaccaattgtcatgcttgacacaactgctgtggctggttggtgctttgtttatggtagtagtttttctgtaacacagaatataggataagaaataagaataaagtaccttgactttgttcacagcatgtagggtgatgagcactcacaattgttgactaaaatgctgcttttaaaacataggaaagtagaatggttgagtgcaaatccatagcacaagataaattgagctagttaaggcaaatcaggtaaaatagtcatgattctatgtaatgtaaaccagaaaaaataaatgttcatgatttcaagatgttatattaaagaaaaactttaaaaattattatatatttatagcaaagttatcttaaatatgaattctgttgtaatttaatgacttttgaattacagagatataaatgaagtattatctgtaaaaattgttataattagagttgtgatacagagtatatttccattcagacaatatatcataacttaataaatattgtattttagatatattctctaataaaattcagaattcta

SEQ ID NO: 3 - protein, 208 aa, synthetic construct
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 4 - DNA, 777 bp, synthetic construct
  source 1..777: mol_type = other DNA
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 5 - protein, 259 aa, synthetic construct
SHHWGYGKHNGPEHWHKDFPIAKGERQSPVDIDTHTAKYDPSLKPLSVSYDQATSLRILNNGHAFNVEFDDSQDKAVHKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 6 - DNA, 616 bp, synthetic construct
  source 1..616: mol_type = other DNA
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtga

SEQ ID NO: 7 - protein, 259 aa, synthetic construct; identical to SEQ ID NO: 5

SEQ ID NO: 8 - DNA, 616 bp, synthetic construct; identical to SEQ ID NO: 6
  source 1..616: mol_type = other DNA

SEQ ID NO: 9 - protein, 159 aa, Homo sapiens [sequence omitted]

SEQ ID NO: 10 - protein, 187 aa, Homo sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 11 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 12 - protein, 327 aa, Homo sapiens
MEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ